(19) United States
(12) Patent Application Publication
(10) Pub. No.: US 2009/0142357 A1
(43) Pub. Date: Jun. 4, 2009

(54) Specific Depletion of CD4+ T Cells

(75) Inventor: Hamid Rabb, Ellicott City, MD (US)

Correspondence Address: BANNER & WITCOFF, LTD., 1100 13th STREET, N.W., SUITE 1200, WASHINGTON, DC, 20005-4051, US

(73) Assignee: THE JOHNS HOPKINS UNIVERSITY, Baltimore, MD (US)

(21) Appl. No.: 12/261,516

(22) Filed: Oct. 30, 2008

Related U.S. Application Data
(62) Division of application No. 10/488,548, filed on Dec. 28, 2004, now abandoned.
(60) Provisional application No. 60/323,001, filed on Sep. 18, 2001.
(60) Provisional application No. 60/386,120, filed on Jun. 5, 2002.

(51) Int. Cl. A61K 39/395; A01N 1/02

(52) U.S. Cl. 424/173.1; 435/1.2

(57) ABSTRACT

Specific depletion or modulation of activity of CD4⁺ T cells is used to treat and/or prevent damage due to ischemia of kidneys, heart, brain. It also has been found to reduce rejection of transplanted organs. The donor, recipient, or isolated organ can be so treated.

This application is a divisional application of Ser. No. 10/488,548 filed Dec. 28, 2004, which is a national stage of International Application No. PCT/US02/29549 filed Sep. 18, 2002, which was published under PCT Article 21(2) in English on Mar. 27, 2003 (International Publication Number WO 03/024399 A2), which claims the benefit of U.S. Provisional Application 60/323,0001 filed Sep. 18, 2001 and U.S. Provisional Application 60/386,120 filed Jun. 5, 2002. The United States of America retains certain rights in the invention by virtue of its grant NIH DK54770.

### FIELD OF THE INVENTION

The invention relates to renal failure, cardiac arrest, cardiac ischemia, organ transplantation and ischemic stroke. In particular, it relates to methods of treatment and prevention of these diseases and conditions.

### BACKGROUND OF THE INVENTION

Ischemic acute renal failure is the most common cause of intrinsic ARF in adults (1). In native kidneys it is associated with an overall mortality rate of up to 50% (2). Despite developments in dialysis treatment, this mortality rate has not improved in the last 30 years (3). In the transplanted kidney, ischemia-reperfusion injury (IRI) is the main cause of delayed graft function (DGF), occurring in 30% of all cadaveric transplants (29). Each case of DGF adds $25,000 to the initial hospitalization of the patient (30). Renal IRI leads to an increased risk of acute rejection, which in turn leads to an increased risk of chronic graft loss (29). There is currently no specific treatment for renal IRI.

Recently, leukocytes have been implicated in the pathogenesis of renal ischemia reperfusion injury (IRI), and most of the work has focused on the role of the neutrophil (5). Evidence from several studies suggests, however, that T cells could also be important leukocyte mediators of renal IRI. Lymphocytes have been found in postischemic human (6, 7) and rat (8) kidneys, particularly in the outer medulla. We recently found that genetically engineered mice deficient in both CD4+ and CD8+ lymphocytes had substantially less kidney dysfunction after renal ischemia than did wild type control mice with renal IRI (9). T lymphocytes have also been found to be important in the pathogenesis of IRI in other organs (10). Targeting the T cell co-stimulatory pathway with CTLA-4Ig has abrogated renal IRI in rats (33). We have recently demonstrated a direct role for CD4 and CD8 T cells in renal IRI using murine model and CD4/CD8 double knockout mice (34). We have also found that nude mice are protected from renal IRI, and that T cell adoptive transfer reconstitutes injury phenotype. CD4−/− mice are significantly protected from renal IRI, and CD4+ T cell adoptive transfer also reconstitutes injury (35). However, observations in mutant mice that are totally devoid of certain T cell populations may not reflect the effect of an intervention in wild type mice or humans. OKT3 is an established T cell depleting antibody (Ab) used to treat human allograft rejection (36). Given the feasibility of treating transplant patients with T cell depleting Abs, it would be useful to know if renal IRI, which occurs during organ procurement and transport during transplant, is also responsive to T cell depletion.

### BRIEF SUMMARY OF THE INVENTION

According to one embodiment of the invention a method is provided for preventing or treating acute renal failure in a patient that is at risk of or experiencing acute renal failure. CD⁴⁺ T cells in the patient are depleted to a level which is less than 5% of the untreated level in the patient's peripheral blood.

According to another embodiment of the invention a method is provided for treating an isolated organ for transplantation. CD4⁺ T cells in blood perfusing the isolated organ are depleted to reduce their level to less than 5% of the untreated level.

In yet another embodiment of the invention a method is provided to treat an organ donor. CD4⁺ T cells in the organ donor are depleted to a level which is less than 5% of the untreated level in the organ donor's peripheral blood.

In still another embodiment of the invention a method to treat a transplant organ recipient is provided. CD4⁺ T cells in the transplant organ recipient are depleted to a level which is less than 5% of the untreated level in the transplant organ recipient's peripheral blood.

Also provided by the present invention is a method to treat or prevent cardiac arrest or ischemia. CD4⁺ T cells in a patient are depleted to a level which is less than 5% of the untreated level in peripheral blood.

Yet another embodiment of the invention provides a method to treat or prevent ischemic stroke. CD4⁺ T cells in a patient are depleted to a level which is less than 5% of the untreated level in peripheral blood.

According to one embodiment of the invention a method is provided for preventing or treating acute renal failure in a patient that is at risk of or experiencing acute renal failure. Activity of CD4⁺ T cells in the patient is modulated such that level of IFN-γ in the patient's peripheral blood is less than 35% of untreated level.

According to another embodiment of the invention a method is provided for treating an isolated organ for transplantation. Activity of CD4⁺ T cells in blood perfusing the isolated organ is modulated to reduce level of IFN-γ to less than 35% of untreated level.

In yet another embodiment of the invention a method is provided to treat an organ donor. Activity of CD4⁺ T cells in the organ donor is modulated such that the level of IFN-γ is less than 35% of untreated level in the organ donor's peripheral blood.

In still another embodiment of the invention a method to treat a transplant organ recipient is provided. Activity of CD4⁺ T cells in the transplant organ recipient is modulated such that the level of IFN-γ is less than 35% of untreated level in the transplant organ recipient's peripheral blood.

Also provided by the present invention is a method to treat or prevent cardiac arrest or ischemia. Activity of CD4⁺ T cells in a patient is modulated such that level of IFN-γ is less than 35% of untreated level in peripheral blood.

Yet another embodiment of the invention provides a method to treat or prevent ischemic stroke. Activity of CD4⁺ T cells in a patient is modulated such that level of IFN-γ is less than 35% of untreated level in peripheral blood.

### BRIEF DESCRIPTION OF THE DRAWINGS

FIG. 1A-D show flow cytometry confirmation of the absence or presence of CD4+ and/or CD8+ T cells. FIG. 1A, CD4+ and CD8+ T cells are absent from spleens obtained from nu/nu mice. (CD4 analysis left panel, CD8 analysis right panel for all) FIG. 1B, After nu/nu mice were adoptively transferred with a T cell population from a wild type control mouse, both CD4+ and CD8+ T cells were detected in spleens. The reconstitution of these cells in nu/nu mice ranged between 2 and 6%. FIG. 1C, CD4+ deficient mice show an absence of CD4+ T cells, however, they do show a normal complement of CD8+ T cells when compared to a wild type control (not shown). FIG. 1D, CD4+ reconstituted CD4+ deficient mice show a CD4+ population of approximately 2%.

FIG. 2 shows T cell deficient mice are functionally protected from postischemic renal injury. SCr was significant reduced at 24 and 48 h (*P<0.05) following IRI in nu/nu mice () compared to SCr in wild type control mice with IRI (▪). The injury phenotype was restored when nu/nu mice were adoptively transferred with wild type T cells (▾). Reconstituted nu/nu mice show a significantly higher SCr at 24 and 48 h postischemia (#P<0.05) compared to nu/nu mice. In all experiments, serum creatinine (mg/dL) was measured from tail blood samples obtained from animals at 0 (preischemia), 24, 48 and 72 h after moderate ischemia (30 min bilateral clamping). T cell transferred mice received 15×10⁶ purified T cells I.P. obtained from wild type control mice 3 weeks prior to ischemic injury.

FIG. 3A-D show histological assessment of tubular injury at 72 h postischemia in T cell deficient and T cell reconstituted mice. FIG. 3A, represents a normal wild type mouse kidney that has not undergone IRI. FIG. 3B, shows a wild type kidney 72 h postischemia and demonstrates extensive tubular damage. nu/nu postischemic kidney shows a significant reduction in renal structural injury (FIG. 3C). In contrast, T cell reconstituted nu/nu mice show a return of postischemic renal injury with similar structural damage as seen in the wild type kidney (FIG. 3D) X50

FIG. 4 shows infiltrating neutrophils into the postischemic kidney at 24 and 72 h postischemia in T cell deficient and T cell reconstituted mice compared to wild type control mice. At 24 and 72 h postischemia there was no significant difference in the amount of infiltrating neutrophils between wild type mice □, nu/nu ▪ and T cell reconstituted nu/nu mice [symbol] N=6 per group.

FIG. 5 shows infiltrating macrophages into the postischemic kidney at 72 h postischemia in T cell deficient and T cell reconstituted mice compared to wild type control mice. At 72 h postischemia there was no significant difference in the amount of infiltrating macrophages between wild type mice □, nu/nu ▪ and T cell reconstituted nu/nu mice [symbol] N=6 per group.

FIG. 6 demonstrates that CD4+ KO mice are functionally protected from renal injury compared to CD8+ KO mice. CD4+ deficient mice (♦) show significantly reduced SCr at all time points when compared to wild type mice (▪) (*P<0.05), and compared to CD8+ deficient (▴) (#P<0.05) mice at 24 and 48 h following renal IRI.

FIG. 7 demonstrates adoptive transfer of CD4+ T cells into CD4+ deficient mice restores injury phenotype. CD4+ deficient mice that have been reconstituted with wild type CD4+ T cells (▾, dashed line) have a return of the injury phenotype, with SCr values similar to wild type control mice (▪). A significant increase in SCr in CD4+ reconstituted mice was seen at 72 h postischemia compared to CD4+ deficient mice (♦) (#P<0.05). CD4+ reconstituted mice received 5-10×10⁶ purified T cells I.P. obtained from wild type control mice 3 wk prior to ischemic injury.

FIG. 8A-8D show histological assessment of tubular injury at 24 h postischemia in CD4+ deficient and CD8+ deficient mice. FIG. 8A, represents a normal wild type mouse kidney that has not undergone IRI. At 24 h postischemia there is severe tubular damage in the wild type control kidney (FIG. 8B). However, the CD4+ deficient mouse kidney shows a significant reduction in tubular injury when compared to the wild type control (FIG. 8C). In contrast, the CD8+ deficient mouse kidney (FIG. 8D) shows tubular injury similar to that seen in the wild type control kidney. X50

FIG. 9 demonstrates infiltrating neutrophils into the postischemic kidney at 24 and 72 h postischemia in CD4+ deficient and wild type control mice. At 24 and 72 h postischemia there is no significant difference seen in the amount of infiltrating neutrophils between wild type mice □, and CD4 deficient ▪. N=6 per group.

FIG. 10 shows infiltrating macrophages into the postischemic kidney at 24 and 72 h postischemia in CD4+ deficient and wild type control mice. At 24 and 72 h postischemia there was no significant difference seen in the amount of infiltrating macrophages between wild type mice □, CD4 deficient ▪. N=6 per group.

FIG. 11A-11B demonstrates infiltrating CD4+ T cells into the postischemic kidney. FIG. 11A, represents a positively stained wild type spleen showing the presence of CD4+ T cells. This is represented by brown rings, which surround positive cells. FIG. 11B, is a wild type (no IRI) kidney stained with an antibody for CD4+ and C is a 24 h postischemic kidney obtained from a wild type control mouse. It shows a small infiltration of CD4+ T cells. X50

FIG. 12A-12C show flow cytometry confirmation of adoptive transfer of CD28 deficient and IFN-γ deficient CD4+ T cells into T cell deficient mice. FIG. 12A, Wild type spleen showing normal numbers of positively stained CD4+ (15.8%) and CD8+ (13.1%) T cells. FIG. 12B, Nu/nu mice were reconstituted with CD4+ T cells (5.5% positive stained) from mice deficient in CD28. FIG. 12C Nu/nu mice were repleted with CD4+ T cells (4% positive stained) from mice deficient in IFN-γ.

FIG. 13 shows adoptive transfer with CD4+ T cells from mice deficient in either CD28 or IFN-γ does not return the injury phenotype. Nu/nu mice [symbol] show significantly reduced SCr at all time points when compared to wild type mice (), nu/nu mice repleted with CD4+ T cells from mice deficient in either CD28 (▪) or IFN-γ (∇) also show significantly reduced SCr at all time points when compared to wild type control mice.

FIG. 14 illustrates the experimental design. Each animal underwent thymectomy or sham surgery for thymectomy. After their recovery from the surgery, i.p. injection of Ab or PBS was performed at the day 14, 16, 18. At the day 21, renal IRI was performed for all animals. Three main groups were studied: 1) Sham thymectomy, PBS i.p. injection, and then renal IRI; 2) Thymectomy, anti-CD4 and CD8 Ab i.p. injection, and then renal IRI; 3) Thymectomy, anti-Thy1.2 (CD90), CD4 and CD8 Ab IP injection.

FIG. 15 shows the effects of T cell antibodies on renal function postischemia. Serum creatinine data demonstrated that thymectomy with triple Ab depletion significantly protected from IRI compared with thymectomy with double Ab depletion or sham for thymectomy (*P<0.05). [X: Sham for thymectomy, : Double Ab Depletion, ◯: Triple Ab depletion]

FIG. 16A-16C show flow cytometry plots. FIG. 16A) Sham thymectomy with PBS IP injection; FIG. 16B) Thymectomy with double Ab depletion demonstrating significant depletion of CD4 and CD8 populations; FIG. 16C) Thymectomy with triple Ab depletion showing a more pronounced depletion of CD4 cells than double Ab treatment.

FIG. 17 demonstrates cell counts of spleen and lymph nodes demonstrating depletion after Ab treatments. CD4 T cells, though significantly depleted after double Ab treatment, were still present in both spleen and lymph nodes. These residual CD4 T cells were further decreased further by after triple Ab depletion. (*, [symbol] P<0.05)

FIG. 18 shows peripheral blood cell counts demonstrating after triple Ab treatment with or without thymectomy. White blood cells, primarily lymphocytes, were successfully depleted. Ab depletion using the combination of mAbs specific to T cells did not affect other leukocytes, such as neutrophils and monocytes. Ab treatment in the absence of thymectomy led to similar leukocyte depletion compared to Ab treatment with thymectomy (*, [symbol] P<0.05)

FIG. 19 shows flow cytometry of T cell counts from peripheral blood after triple Ab treatments-with or without thymectomy. Ab treatment successfully depleted peripheral blood CD4 as well as CD8 T cells. Thymectomy did not influence the effect of Ab depletion. (*, [symbol] P<0.05)

FIG. 20A-20C shows renal histology at 72 hours postischemia; FIG. 20A) In animals with sham thymectomy who had PBS injection before IRI, extensive tubular necrosis with cast formation and epithelial cell sloughing is seen. FIG. 20B) Double Ab depletion did not significantly improve structural damage after IRI; FIG. 20C) Improved tubular architecture after IRI in mice with triple Ab depletion (hematoxylin-eosin stain, 400× magnification).

FIG. 21 shows tubular injury score of mice after IRI. Triple Ab depletion resulted in significantly lower tubular injury scores compared with sham or double Ab treated group (*P<0.05).

### DETAILED DESCRIPTION OF THE INVENTION

Ischemia-reperfusion injury (IRI) is the main cause of acute renal failure in both allograft and native kidney. Studies using T cell knockout mice have established an important role for T cells in renal IRI. T cell depletion strategies are effective in human allograft rejection. However, it is not known whether those are effective in renal IRI. We therefore studied the effect of T cell depletion in a murine model of renal IRI using well-characterized antibodies that have been effective in preventing experimental allograft rejection. These data demonstrate that T cell depletion can improve the course of experimental renal IRI. However, more aggressive T cell depletion strategies were required compared to that needed to prevent experimental allograft rejection.

We tested this using an established model of renal IRI (34). We used antibodies that have been well characterized in mouse models to deplete T cells and prevent allograft rejection. We found that when these antibodies were used individually, no protection was afforded from renal IRI. However, when administered together, there was more effective T cell depletion that in turn was associated with a significant functional and structural protection to the kidney after I/R. Thus in experimental renal IRI, in contrast to allograft rejection models, even small numbers of T cells may be sufficient to mediate significant tissue injury.

CD4⁺ T cells can be depleted using techniques known in the art for doing so. These include administration of antibodies which are specific for T cells and administration of antibodies that are specific for CD4⁺ T cells or subsets thereof. In order to achieve therapeutic or prophylactic levels of CD4⁺ T cells, it may be necessary to use more than one antibody. Cocktails of antibodies are contemplated to achieve effective depletion. The antibodies can be specific for different antigens on T cells or on CD4⁺ T cells. Antibodies specific for CD4, CD8, and Thy 1.2 (CD90) antigens may be particularly useful. Other agents which specifically deplete CD4⁺ T cells can also be used. Effective levels of depletion for treating patients at risk of or in the midst of acute renal failure are less than 10%, less than 7.5%, less than 5%, less than 4%, less than 3%, less than 2%, or less than 1% of pretreatment levels. Patients at risk of acute renal failure include but are not limited to those with diabetes, hypertension, chronic glomerulonephritis, and polycystic kidney disease.

Blood perfusing an isolated organ can also be treated to deplete CD4⁺ T cells.

Suitable transplantable organs for treatment include liver, intestine, heart, lung, and brain. Preferably the level is reduced to less than that of the untreated level. Similarly, both organ donors and organ recipients can be so treated. The recipient can be treated before, after, or during transplantation. Effective levels of depletion are less than 10%, less than 7.5%, less than 5%, less than 4%, less than 3%, less than 2%, or less than 1% of pretreatment levels. Such treatment has been found to provide both a short term and a long term benefit.

Patients susceptible to or in the midst of cardiac arrest or ischemia may be similarly treated to deplete CD4⁺ T cells. Such treatment protects against reperfusion injury. Patients who are susceptible to or at risk of cardiac arrest or ischemia include those in shock, those who are bleeding, those who have taken an overdose of a drug, and those who have heart disease, including angina. In addition, patients susceptible to or in the midst of ischemic stroke may be similarly treated. Such patients include but are not limited to those having early symptoms of stroke, those about to undergo high-risk surgery, and those with vascular disease.

As an alternative to a depletion strategy, CD4⁺ T cells can be treated to modulate their activity. Preferably they are treated such that level of IFN-γ in the patient's peripheral blood is less than 35% of untreated level. IFN-γ can also be assessed in the patient's T cells or tissue, e.g., spleen and lymph nodes. Cytokine IL-12 can also be used as an index of modulation. The level of IFN-γ or IL-12 can be advantageously diminished to achieve pre-treatment or non-treatment levels of less than 30%, less than 25%, less than 20%, less than 15%, less than 10%, less than 5%, or less than 2%. Similar classes of patients and organs can be treated for modulation as for depletion. Agents which can be used for modulation include, but are not limited to tamoxifen and the MDR1 P-gp-specific mAb Hyb-241. Frank et al., J. Immunol. 2001 166:2451-9. Another agent which can be used is TJU103, a small organic molecule which was selected by computer screening. TJU103 is a small nonpeptidic molecule that blocks the interaction between major histocompatibility complex class II and CD4 molecules. Krakauer, Antimicrob. Agents Chemother. 2000 44:1067-9. TJU103 is N-(3-indoylmethylene)-isonicotinic hydrazone. Li et al., Proc. Natl. Acad. Sci. USA 1997 94:73-8. See also TJU101, 102, and 104. It specifically inhibits CD4⁺ T cells by disrupting the function of the CD4 molecule during activation. Edling et al., J. Autoimmuni. 2002 18:169-79.

Antibodies, small organic molecules, or other agents for depletion of CD4⁺ T cells or for modulation of their activity can be administered by any means known in the art. Typically such agents will be injected or infused intravenously, although other routes of administration are possible. Other routes include, without limitation, intraperitoneal, intramuscular, transdermal, subcutaneous, per os. Direct administration to the spleen, thymus, or lymph nodes, sites of T cell production, maturation, or concentration, can also be used. Such agents can also be administered directly to an involved organ, such as a transplanted or diseased organ at risk of ischemia, such as the liver, kidney, intestine, heart lung, or brain.

### EXAMPLE 1

We hypothesized that the T cell has an important role in the pathogenesis of renal IRI, and sought to directly identify which T cell subset might be more important. The hypothesis was investigated in a redundant approach using distinct strains of T cell deficient mice and adoptive transfer techniques. T cell deficient (nu/nu) mice were found to be protected from postischemic renal injury, while nu/nu reconstituted with wild type T cells were found to have postischemic renal injury similar to that in wild type mice. To elucidate the primary T cell subset important in renal IRI, mice deficient in CD4+ T cells alone or CD8+ T cells alone were subjected to renal IRI. The CD4+ deficient mice had significantly improved recovery of renal function postischemia compared to CD8+ deficient mice and wild type control mice. Furthermore, CD4+ deficient mice reconstituted with CD4+ T cells had a restoration of the postischemic injury phenotype. To identify a possible mechanism involved in the protection from renal function seen in the T cell deficient mice, we studied nu/nu mice that had been adoptively transferred with an enriched CD4+ T cell population obtained from mice that were deficient in either CD28 (accessory molecule mediated pathway) or interferon gamma (IFN-γ) (to distinguish between a possible Th1 versus a Th2 response). We found that both CD28 and IFN-γ are important components of the CD4+ mediated renal injury following IRI, as neither defective CD4+ population restored the postischemic injury phenotype.

### Methods

### Mice.

Nu/nu mice (B6.Cg-Foxn1^{nu}) and C57BL/6 wild type littermates were purchased from Jackson Laboratories (Bar Harbor, Me.). The two main defects of mice homozygous for the nu/nu spontaneous mutation (Foxn1^{nu}, formerly Hfh11^{nu}) are abnormal hair growth and defective development of the thymic epithelium. Nu/nu mice are therefore athymic due to a developmental failure of the thymus. Consequently, homozygous nu/nu mice lack T cells and cell-mediated immunity. CD4+ deficient mice (B6.129S2-CD4+^{tm1mak}) and CD8+ deficient mice (B6.129S2-CD8+^{tm1mak}), CD28 deficient mice (B6.129S2-Cd28^{tm1Mak}), IFN-γ deficient mice (B6.129S7-Ifng^{tm1Ts}) and wild type littermates were also purchased from Jackson Laboratories. Mice were housed under pathogen free conditions at the Minneapolis Medical Research Foundation Animal Facility, according to NIH guidelines. To confirm knockout and adoptive transfer status, spleens and lymph nodes from each group of mice were collected upon sacrifice and analyzed for CD4+ and CD8+ cells using flow cytometric analysis.

### Renal Ischemia Reperfusion Model

An established model of renal IRI was used (9). Animals were anesthetized with sodium pentobarbital (75 mg/kg, I.P.). Abdominal incisions were made and the renal pedicles were bluntly dissected. A microvascular clamp was placed on both renal pedicles for 30 min. An ischemia time of 30 min was chosen because it produces a sublethal kidney injury with many similarities to that seen in human IRI with acute tubular necrosis. During the procedure, animals were kept well hydrated with saline and were kept at a constant temperature (˜37° C.). After the clamps were removed, the wounds were sutured and the animals were allowed to recover.

### Assessment of Postischemic Renal Function

Blood samples were obtained from the tail vein at 0, 24, 48 and 72 h postischemia. Serum creatinine (mg/dL) was measured on a Cobas Fara automated system (Roche, Nutley, N.J.) using a Creatinine 557 kit (Sigma Diagnostics, St Louis, Mo.)

### Tissue Histological Examination

At 24 or 72 h postischemia, kidneys were dissected from mice and coronal tissue slices were fixed in 10% formalin and processed for histology using standard techniques. Formalin tissue was embedded in paraffin and 4 μm slices were cut. Basement membranes and other oxidizable 1,2-diol containing structures were visualized using Periodic acid-Schiff (PAS). These were examined in a blinded fashion to determine the extent of tubular necrosis.

### Immunohistochemistry

Tissue sections were prepared as described above for routine histology. Sections (4 μm) were prepared on a cryostat and mounted on Fisher Superfrost plus slides, fixed in ice cold acetone for 1-2 min and allowed to air dry. Sections were then blocked with 1:100 normal rabbit serum in PBS containing Vector avidin DH (Vector Laboratories Inc. Burlingame, Calif.). Primary antibodies were then added to the sections; GK 1.5 (rat anti-mouse CD4 (ATCC, Manassas, Va.)), GD 2.43 (rat anti-mouse CD8 (ATCC, Manassas, Va.)), M1/70 (rat anti-mouse MAC-1 (Caltag, Burlingame, Calif.)) and 7/4 (rat anti-mouse neutrophil (Accurate Chem. And Scientific Corp., Westbury N.Y.)) and incubated for 1 h at room temperature. Background staining controls consisted of an isotype control primary antibody. Sections were then rinsed in PBS and treated with 3% hydrogen peroxide in Biotin (10 μg/L PBS) to block the biotin binding sites. After three washes in PBS, the slides were incubated in a biotin-conjugated rabbit anti-rat IgG secondary antibody (Vector Laboratories Inc. Burlingame, Calif.) for 35 min at room temperature. Sections were once again washed and incubated for 45 min in Vector Elite ABC (Vector Laboratories Inc. Burlingame, Calif.). Sections were washed and developed with 3-amino-9-ethyl-carbizole, counterstained with hematoxylin and mounted using Glycergel (Dako, Carpinteria Calif.). After viewing the entire kidney section, ten high powered (X40) fields were counted in the area of corticomedullary junction and total numbers of cells were quantified in a blinded fashion.

### T Cell Adoptive Transfer

Spleens and lymph nodes were collected from C57BL/6 wild type littermates, CD28 deficient mice or IFN-γ deficient mice. Splenic cells were collected by centrifugation and the red blood cells were removed by lysis in NH4Cl for 5 min. T cell enrichment was performed using nylon wool column chromatography. Briefly, sterile nylon wool packed columns were equilibrated in RPMI with 5% newborn calf serum and cells were incubated on the column for 1 h at 37° C. in 5% CO2. Nonadherent cells (predominantly T cells) were then eluted in 15 ml and collected by centrifugation. The percentages of CD4+ and CD8+ T cells were assessed in pre- and post-nylon wool T cell-enriched fractions by flow cytometry. Enriched T cells were then washed 3 times in HBSS to remove serum from the preparation. Approximately 15×106 enriched T cells were injected I.P. into each nu/nu mouse. IRI was induced in the mice 3 wk post transfer. The 3 wk time point was based on our preliminary studies in which earlier time points had less efficient reconstitution.

Purified populations of CD4+ T cells were obtained by negative selection of CD8 T cells and B cells on an immunoaffinity column according to the manufacturer's specifications (Cellect-plus kits, Cytovax Biotechnologies Inc., Alberta, Canada). Enriched CD4+ T cells were eluted from each column and approximately 5-10×10⁶ were injected I.P. FACS analysis was performed before and after column purification to determine percentages of CD4+ and CD8+ in cell preparations. Ischemia was performed on the mice 3 wk post transfer.

### Flow Cytometry (FACS) Analysis

Postischemic spleens and/or lymph nodes were crushed in 5 ml HBSS-2 using 5 strokes in a glass homogenizer. Cells were collected by centrifugation at 250 g for 10 min. They were then re-suspended in ammonium chloride potassium solution (ACK buffer) for 5 min to remove red blood cells. Cells were washed 3 times with ice cold HBSS-2 and filtered through a 70 μm nylon screen to remove debris. For each analysis, 10⁶ cells were treated with a Fc block for 5 min then stained with 0.5 μg R-PE rat anti-mouse CD4 (H129.19) antibody and FITC anti-mouse CD8b.2 (53-5.8) antibody or isotype controls (Pharmingen, CA) for 1 h on ice. Cells were then washed twice, fixed in 1% formalin, and analyzed on a Epics XL flow cytometer using System II software (Coulter Corp., FL).

### Statistical Analysis

Data are expressed as mean±standard error. Comparisons of group means were performed using a one-way analysis of variance with the Student-Newman-Keuls multiple group comparison test. A P<0.05 was considered significant.

### Results

### Confirmation of T Cell Knockout and Repletion Status

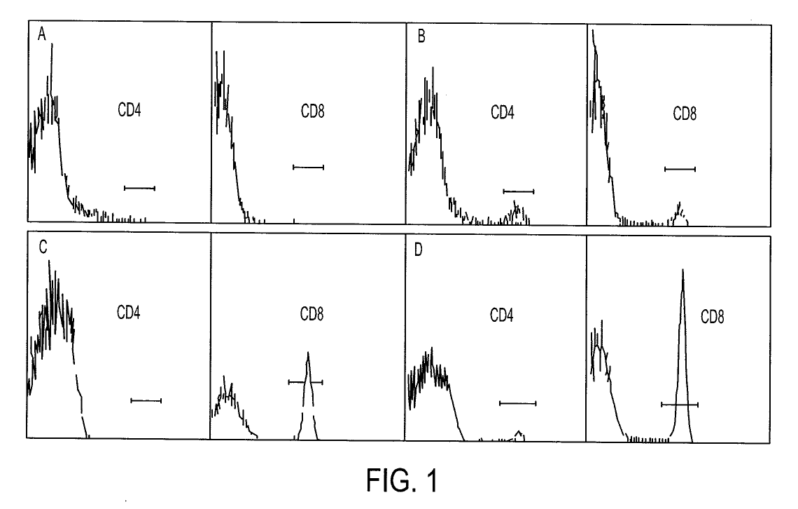

To confirm the absence or presence of CD4+ and/or CD8+ T cells, we used flow cytometry to analyze lymph nodes and spleen from each mouse upon sacrifice. FIG. 1A shows the absence of both CD4+ and CD8+ cells in nu/nu mice, the presence of CD4+ and CD8+ cells in T cell reconstituted nu/nu mice (FIG. 1B), the absence of CD4+ cells in CD4+ deficient mice (FIG. 1C) and the presence of CD4+ T cells in CD4+ deficient mice that were reconstituted with CD4+ cells (FIG. 1D). CD4+ cells that were taken from mice either deficient in either CD28 or IFN-γ were equivalent to wild type cells in their ability to reconstitute after adoptive transfer. FACS analysis was also used to determine the enrichment of T cells in the adoptive transfer experiments. Nylon wool was used to obtain an enriched population of T cells from a lymphocyte cell preparation. Pre nylon wool FACS analysis showed CD4 positive staining of 11.4% and CD8 positive staining of 7.75%. Following nylon wool enrichment these values were increased; 36.2% CD4+ positive stained cells and 39.7% CD8+ positive stained cells. For the CD4+ adoptive transfer experiment, the pre CD4+ enrichment column cell preparation consisted of 19.1% CD4+ positive cells. Post CD4+ enrichment column FACS analysis resulted in an increase of CD4+ positive cells to 71.8%.

### T Cell Deficient Mice Show a Return of the Injury Phenotype When Adoptively Transferred With an Enriched T Cell Population.

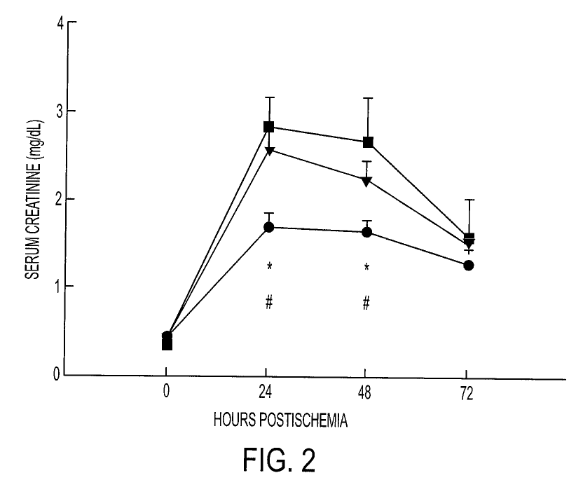

To demonstrate the effect of T cell deficiency on early renal dysfunction following IRI, we first evaluated renal function postischemia in nu/nu mice. These mice underwent moderate renal ischemia (30 min renal artery clamping) and were monitored for 72 h postischemia. Postischemic renal function in nu/nu mice and wild type control mice is shown in FIG. 2. Serum creatinine (SCr) was significantly reduced at 24 and 48 h postischemia in the nu/nu mice, when compared to wild type control mice with IRI (n=8, 1.73±0.19 vs 2.86±0.13, *P<0.05) and (n=8, 1.67±0.15 vs 2.7±0.09, *P<0.05). These data demonstrate that T cell deficient nu/nu mice had less renal dysfunction following IRI than did wild type control mice.

Nu/nu mice are genetically mutant mice, and thus may have other differences besides lack of T cells, when compared with wild type control mice. To confirm that reduced ischemic injury in nu/lnu mice was due to the deficiency in T cells, we investigated whether reconstituting nu/nu mice with T cells from wild type mice would return the renal IRI phenotype. An adoptive transfer technique was used to reconstitute the nu/nu mice with wild type T cells. Postischemic renal function in the T cell reconstituted nu/nu mice is also shown in FIG. 2. A return of the renal IRI phenotype is evident, as SCr was significantly elevated when compared to the nu/nu mice at 24 h (n=8, 1.73±0.19 vs 2.59±0.24, #P<0.05) and 48 h (n=8, 1.67±0.15 vs 2.27±0.25, #P<0.05) postischemia. Moreover, SCr in T cell reconstituted nu/nu mice with IRI was comparable to that in wild type control mice with renal IRI.

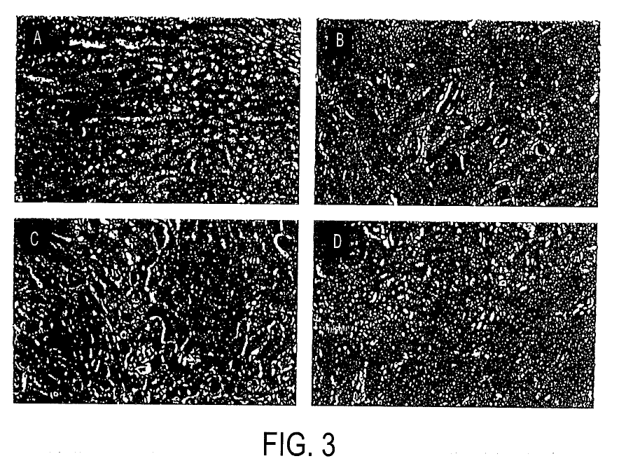

To investigate the extent of tubular necrosis in nu/nu mice, we histologically examined kidney tissue at 24 and 72 h postischemia. Nu/nu mice at 24 h postischemia showed minimal tubular injury (data not shown). We therefore compared 72 h postischemic tissue. Representative photomicrographs are shown in FIG. 3. Normal kidney (no IRI) is represented in FIG. 3A. Kidneys from wild type mice at 72 h postischemia (FIG. 3B) exhibited significant tubular injury, characterized by extensive tubular epithelial necrosis and sloughing of epithelial cells into the tubular lumen. Many of the tubules were dilated. Tubules also contained proteinaceous casts.

Kidneys from nu/nu mice (FIG. 3C) had less tubular injury than did kidneys from wild type control mice (FIG. 3B) at 72 h. In nu/nu mice, a few tubules showed dilatation with proteinaceous material in the lumen. Nu/nu mice that were reconstituted with T cells (FIG. 3D) had more severe tubular injury at 72 h, similar to that seen in wild type mice, with many tubules exhibiting dilation and congestion with proteinaceous material as well as a loss of epithelial cell structure.

### Leukocyte Infiltration Following Ischemia Injury in T Cell Deficient Mice and Reconstituted T Cell Deficient Mice.

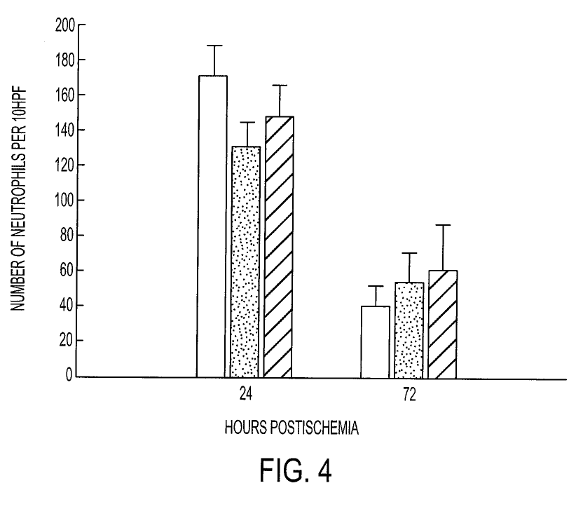

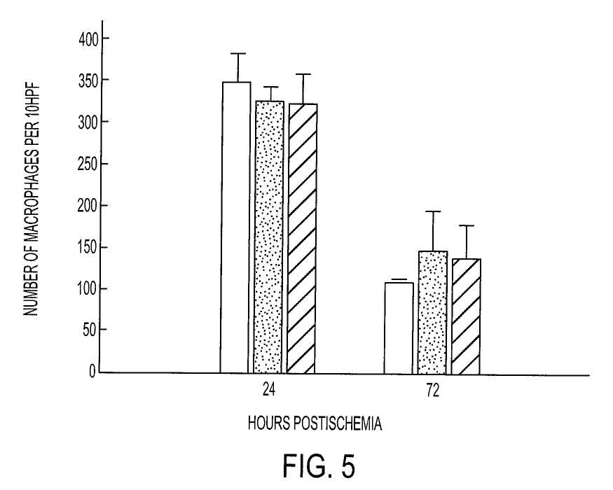

Many studies have proposed that neutrophils play an important role in the postischemic stage of ischemia reperfusion injury, while others have not (5). We quantified the infiltrating neutrophils with a specific antibody at 24 and 72 h postischemia in wild type control, nu/nu and T cell reconstituted nu/nu mice and the results are represented in FIG. 4. Our results show significant neutrophil infiltration in both wild type, nu/nu and T cell reconstituted nu/nu mice. Thus in the current study, it does not appear that neutrophil infiltration into the kidney is correlated with T cell mediated-renal injury. We also analyzed the infiltration of macrophages in the postischemic kidney. Macrophage infiltration, prominent postischemia, was also not significantly different between the T cell deficient nu/nu mice and wild type controls (FIG. 5).

CD4+ Deficient Mice are Functionally and Structurally Protected from Postischemic Renal Injury

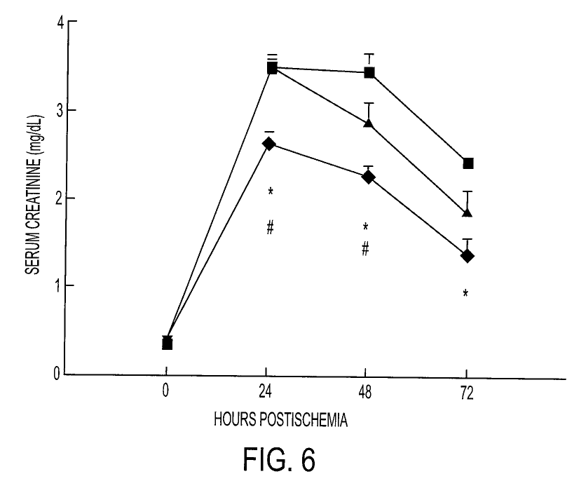

After demonstrating a direct role for T cells in our model of renal IRI, we focused on identifying the important T cell subset involved in the pathogenesis of renal IRI. Mice deficient in either CD4+ T cells alone or CD8+T cells alone were subjected to renal IRI and postischemic structure and function was analyzed. SCr values at 24, 48 and 72 h postischemia are shown in FIG. 6. CD4+ deficient mice had significantly decreased SCr when compared to wild type control mice at each time point postischemia (n=8, *P<0.05). CD4+ deficient mice also had significantly decreased SCr when compared to CD8+ deficient mice at 24 h (n=8, 2.67±0.12 vs 3.48±0.14, #P<0.05) and 48 h postischemia (2.3±0.1 vs 2.89±0.19, #P<0.05). In contrast, CD8+ deficient mice did not show significant improvement in renal function postischemia when compared to wild type control mice. CD4+ deficient mice were also significantly protected from mortality following renal IRI. CD4+ deficient mice showed 88% survival compared to 20% in wild type controls at 72 h postischemia (P<0.05) (data not shown). Taken together, these results demonstrate that it is the CD4+ T cell that is an important T cell subset involved in the pathogenesis of renal injury following IRI.

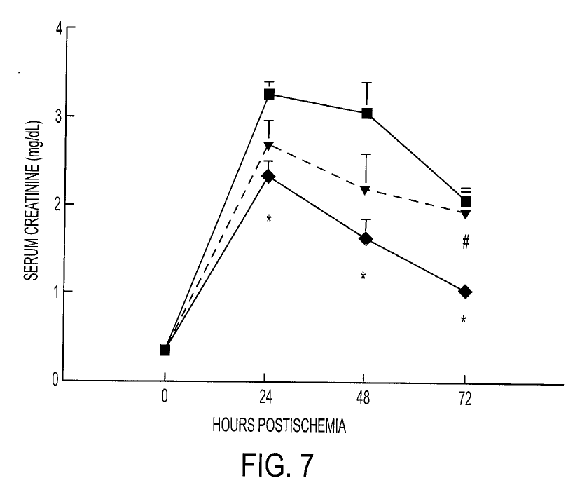

To verify that the lack of CD4+ T cells was responsible for protection from renal IRI, we adoptively transferred CD4+ T cells from wild type control littermates into CD4+ deficient mice. SCr values for these mice are shown in FIG. 7. CD4+ deficient mice were significantly protected compared to wild type mice at each time point (n=8, *P<0.05). The transfer of CD4+ T cells into CD4+ deficient mice restored the IRI phenotype with a significant increase in SCr at 72 h postischemia when compared to that in CD4+ deficient mice (n=8, 1.06±0.07 vs 1.95±0.25, #P<0.05).

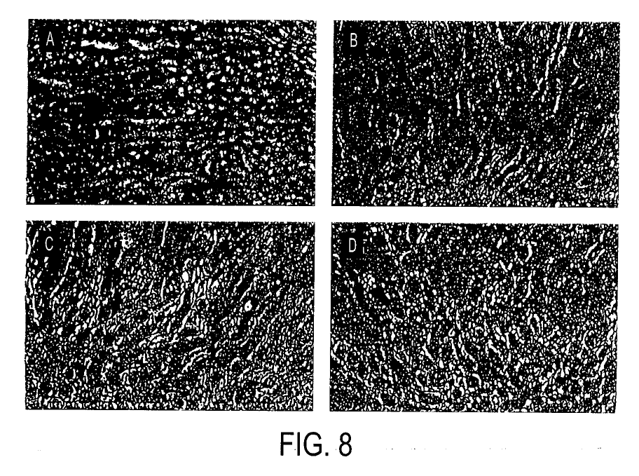

Histological assessment of tubular necrosis demonstrated that the CD4+ deficient mice had reduced renal structural injury. FIG. 8A shows a wild type (no IRI) kidney. At 24 h postischemia there was already a distinct protection from structural injury seen in CD4+ deficient mice kidney (FIG. 8C) when compared to wild type control mouse kidneys at 24 h (FIG. 8B). In contrast, the CD8+ deficient mouse kidneys at 24 h postischemia (FIG. 8D) showed injury similar to that in the wild type control mice.

### Leukocyte Infiltration Into the Postischemic Kidney of CD4 Deficient and Wild Type Mice.

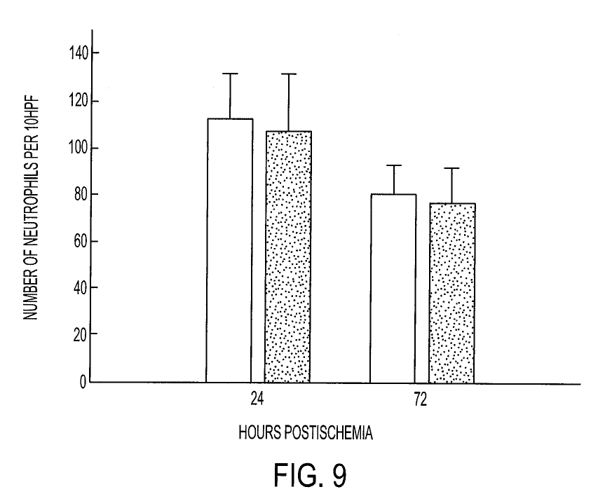

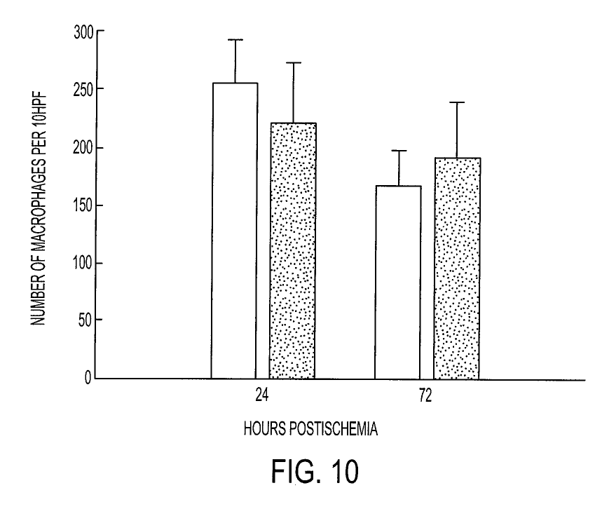

Neutrophil and macrophage infiltration into postischemic kidneys was quantified at 24 and 72 h postischemia in CD4+ deficient and wild type control mice and is represented in FIGS. 9. and 10. These results were similar to those obtained for the T cell deficient nu/nu mice experiments, demonstrating no significant difference in the amount of infiltrating neutrophils and macrophages between these groups.

### CD4 T Cell Infiltration Into the Postischemic Kidney

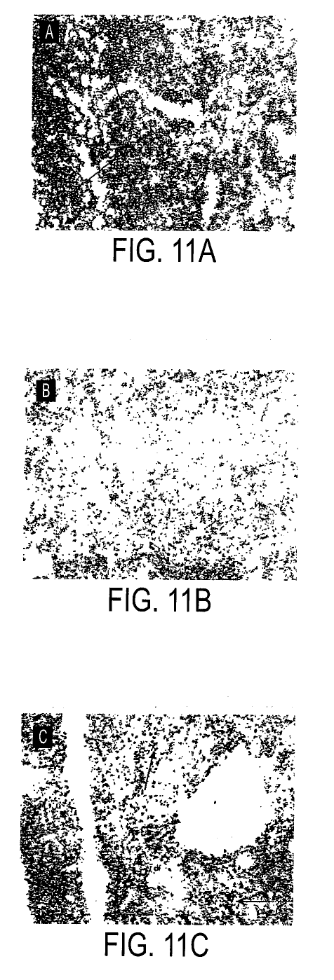

Since our results demonstrate the importance of the CD4+ T cell in influencing the course of renal IRI, we stained postischemic kidney tissue to evaluate a potential infiltration of CD4+ T cells. CD4+ T cell staining is abundant in spleens (FIG. 11A) and is represented by a brown stained ring around positive cells. FIG. 11B shows a normal (no IRI) wild type kidney showing no positive staining for CD4+ T cells. Nu/nu mice, along with the CD4+ knockout showed no positive staining for CD4+ T cells in postischemic kidney. The 24 h wild type postischemic kidney (FIG. 11C) did however show a small number of CD4+ T cells in the outer medulla.

### The Role of CD28 and IFN-γ in T Cell Mediated Renal IRI

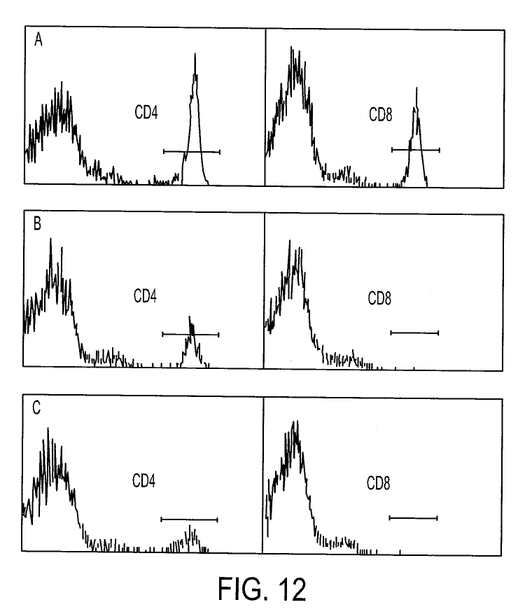

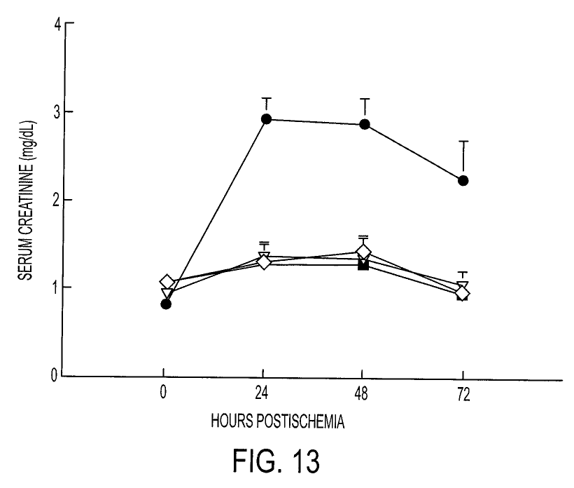

To begin to explore the mechanism of how CD4+ T cells mediate renal IRI, we studied nu/nu mice that had been adoptively transferred with an enriched CD4+ T cell population obtained from mice that were deficient in either CD28 (accessory molecule mediated pathway) or IFN-γ (to distinguish between a possible Th1 versus a Th2 response). FIG. 12 demonstrates the successful adoptive transfer of nu/nu mice with CD4+ T cells from CD28 deficient mice (FIG. 12B) and IFN-γ deficient mice (FIG. 12C). Wild type numbers of CD4+ positive cells and CD8+ positive cells are shown in 12A. Postischemic renal function in nu/nu mice adoptively transferred with CD4+ T cells from CD28 and IFN-γ deficient mice is represented in FIG. 13. Wild type mice show an increase in SCr as previously demonstrated. Also as previously found, nu/nu mice had a significant reduction in serum creatinine compared to that in wild type mice. Adoptive transfer of nu/nu mice with either CD4+ T cells from CD28 deficient mice or CD4+ T cells from IFN-γ deficient mice caused no increase in serum creatinine compared to nu/nu mice. Thus, adoptive transfer of T cells from either CD28 deficient or IFN-γ deficient mice did not produce a return of the injury phenotype.

### Discussion

The current study provides direct evidence that T cells are mediators of renal IRI in mice, and that the CD4+ T cell is the major T cell subset responsible for renal injury following ischemia. Furthermore, the pathophysiologic role of the CD4+ cell is dependent on CD28 and IFN-γ. These results, though novel in the kidney, are consistent with emerging studies in other organ systems. CD4+ T cells have been shown to mediate murine liver ischemic injury in studies of T cell deficient mice (10). A recent study in a model of gut ischemia reperfusion, showed that reconstitution of T cell deficient mice with T-cell enriched splenocytes allowed mice to respond to gut ischemia reperfusion in a manner similar to wild-type mice (11). Using techniques similar to those used in our study, a recent study in a liver model of cold ischemia reperfusion showed that tissue injury was reduced by 51% in nu/nu mice and that injury was restored by T-cell transfer into nu/nu mice (12).

A potential limitation with the use of mutant mice is that physiologic and structural changes observed in IRI could be due to abnormalities independent of the derangement one wants to study. To confirm that T cell deficiency was responsible for reduced renal IRI, we used an adoptive transfer technique to reconstitute both the T cell deficient mice and the CD4+ deficient mice with their respective T cell population. An important and intriguing finding of the present study was that a return of injury phenotype in T cell deficient mice was accomplished with a small degree of T cell reconstitution. Indeed, only a 2-6% splenic reconstitution of T cells was sufficient to restore renal dysfunction and damage. All T cell transferred populations were verified by FACS analysis both prior to cell transfer and at the conclusion of the experiment to ensure minimal effect of contaminating cells. While our results indicate that CD4+ T cells are critical for the pathogenesis of ischemic ARF, they do not exclude the possible contribution of other T cells (eg. CD8+ cells) to the development of IRI. The present results, however, indicate that these cells are not likely the main mediators of early IRI (13, 14).

The mechanism by which CD4+ T cells might induce postischemic renal injury is not known, and two potential pathways were studied. CD4+ T cells from CD28 deficient mice were adoptively transferred into nu/nu mice to identify if accessory molecule expression was important in T cell mediated renal IRI. Our results suggest that CD28 is important in the pathogenesis of renal IRI since our experiments showed that adoptive transfer of nu/nu mice with CD4+ T cells deficient in CD28 was unable to restore injury. The important role for CD28 was hypothesized by Takada et al, who had previously demonstrated that use of CTLA4Ig, which can block CD28 binding to CD80, is protective in a rat model of cold renal IRI (33).

We also examined the effects of reconstituting CD4+ T cells from IFN-γ deficient mice into nu/nu mice. This maneuver was designed to identify if CD4+ cell mediated renal IRI was associated primarily with Th1 or Th2 response. We found that IFN-γ deficient CD4+ T cells did not restore injury in nu/nu mice. This suggests that the Th1 polarization of CD4+ cells might be more important in mediating renal IRI.

T cell activation in renal IRI would not be conventionally expected. Classically, T cell activation is thought to require foreign antigens bound to self-MHC molecules together with costimulatory signals by antigen presenting cells. Nonetheless, T cell activation and the production of associated cytokines have been described in experimental renal IRI (5, 8, 15). The absence of “foreign” antigens in renal IRI leads us to speculate that alloantigen-independent T cell activation may be involved in renal IRI. In this regard, hypoxia itself might be sufficient for T cell activation. CD4+ T cells have been shown to increase adhesion to endothelial monolayers following anoxia/reoxygenation (16). Recently it has also been suggested that increased adhesion of infiltrating T cells to renal tubular epithelial cells under hypoxic conditions may provide a potential mechanism underlying postischemic tubular dysfunction (9). The “danger” signals activated in renal IRI could thus activate the CD4+ T cell and lead it to function in an “innate” manner rather than in its classical “adaptive” immunologic role (17, 18).

Localization of T cells in the kidney would help elucidate the mechanism of the T cell induced renal dysfunction. However, immunohistochemistry with specific antibodies to CD4+ T cells, demonstrated a paucity of T cell infiltration into the postischemic kidney. It may be possible that only a small number of infiltrating T cells, relative to the amount of neutrophil or macrophage infiltration, is sufficient to exert an effect. Alternatively, the T cells could be orchestrating an effect on renal injury from a distant site, possibly via soluble cytokines. In view of previous work, suggesting an important role of neutrophils in renal IRI (19, 20), we investigated in the present study the effect of T cell manipulation on neutrophil infiltration. We have previously used methods such as Leder staining and myeloperoxidase assays to identify neutrophils (21, 22). A recent study, however, has shown that these techniques also detect macrophages and are non-specific (23). In the current study, we therefore used a specific monoclonal antibody to neutrophils to detect their infiltration into postischemic kidney tissue. Brisk neutrophil infiltration to the corticomedullary junction postischemia was observed in both wild type mice, with full expression of renal injury, and T cell manipulated mice that were protected. Similarly, we found significant macrophage infiltration postischemia, with little difference between wild type and T cell deficient groups. Thus, we cannot clearly invoke the neutrophil or macrophage as distal mediators of the T cell in this model. This, however, does not exclude an important role for neutrophils or macrophages in renal IRI. In a previous study, which evaluated leukocyte migration in experimental asthma, we found that leukocyte blockade can protect tissue structure and function despite not attenuating leukocyte migration and infiltration (24, 25).

This study demonstrates the important role for CD4+ T cells in renal IRI using redundant approaches including the use of spontaneous T cell deficient mice, targeted T cell deficient mice, mice with targeted T cell dysfunction, and adoptive transfer techniques. Although many underlying mechanisms still need to be elucidated, our current studies support targeting CD4+ T cells in renal IRI in humans, particularly in the transplant patient.

### EXAMPLE 2

### Materials and Methods

### Animals

Seven to eight-week-old male C57BL/6 mice were obtained from Jackson Laboratory (Bar Harbor, Me.).

### Adult Thymectomy

Thymectomy was performed in all animals as a standard technique to reduce new thymic emigrants after anti-T cell Ab (37). The procedure of mice thymectomy is described elsewhere in depth (13). Briefly, mice were anesthetized by a single intraperitoneal injection of pentobarbital sodium (65 mg/kg). Under sufficient anesthesia, mice were placed on their back and legs were restrained by placing them under parallel rubber bands. An insulin syringe was placed under the neck and the head secured using a rubber band placed across the mouse. Scissors were used for a midline longitudinal skin incision, and sewed roundly in advance. The sternum was then cut laterally from the sternal notch to the third rib. A curved forceps was inserted into the chest cavity to expose the thymus. Using a second set of curved forceps, the thymus was carefully grasped and removed from the chest cavity. For sham surgery, thymus was not removed, just bluntly dissected from the muscles in front of the thymus. The skin was quickly closed by pulling and tying up the sewing thread. Animals were observed under veterinary care for two weeks after surgery to ensure full recovery prior to renal IRI.

### Renal IRI

An established model of renal IRI in mice was used (34). Briefly, 25-35 g mice were anesthetized with intraperitoneal injection of pentobarbital sodium (75 mg/kg), had abdominal incisions, and had the renal pedicles bluntly dissected. Microvascular clamps were placed on the renal pedicles for 30 minutes while the animal was kept at constant temperature and well hydrated. After 30 minutes, the clamps were removed and the wounds sutured, and the animals were allowed to recover. Serum creatinine levels were analyzed at the time of before surgery, 24, 48 and 72 hours postischemia. All animals were sacrificed at the time point of 72 hours postischemia, with kidneys, spleen and lymph nodes harvested.

### Antibodies

The following mAbs directed to mouse T cell surface antigens were obtained from the supernatant of cultured hybridomas maintained in our laboratory: GK1.5 (anti-CD4; American Type Culture Collection (ATCC), Rockville, Md.); 2.43 (anti-CD8; ATCC); 30.H12 (anti-Thy1.2; ATCC)(14,15). Thy1.2 is expressed at the surface of mice peripheral T cells and totally distinct from Thy1.1 which causes experimental glomerulonephritis. Extensive preliminary studies were performed with different dosing and intervals of administration with each Ab to ensure that saturating amounts of Ab were used to deplete T cells. Intraperitoneal injection of each mAb (0.2 mg in 0.2 mL PBS for each animal) was started two weeks after thymectomy and given every other day for 3 doses until the day of ischemic injury. For the group of sham thymectomy, 0.2 mL PBS was injected as a vehicle.

### Experimental Groups

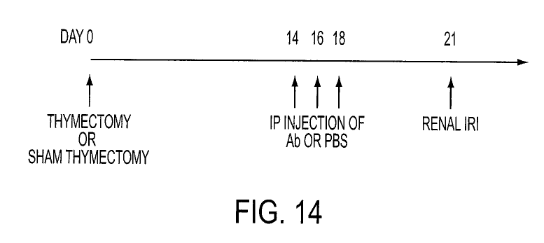

Preliminary studies were performed to ensure that thymectomy lacked significant effect on the course of renal IRI. In addition, preliminary studies were also conducted on mice given individual T cell depleting antibodies in saturating had minimal protective effect on the course of renal IRI. This led the final experimental design with three main experimental groups (n=11/each group); 1) Sham-thymectomy, PBS IP injections, then renal IRI; 2) Thymectomy, double (anti-CD4 and CD8) Ab depletion, then renal IRI; 3) Thymectomy, triple (anti-CD4, CD8 and Thy1.2) Ab depletion, then renal IRI. (FIG. 14).

### T Cell Quantification

T cells were isolated from spleen, lymph nodes and peripheral blood, and were stained by two colors FACS analysis using PE-conjugated mAb to CD4 and FITC-conjugated mAb to CD8b (BD PharMingen, San Diego, Calif.). These detection Abs were different from the depleting Abs in order to prevent potential competition for epitope binding sites which could affect interpretation of the FACs data. Hemocytometer was used to count total cell numbers. After staining, cells were fixed with 1% formaldehyde, and were analyzed by flow cytometry using FACS scan (Beckman Coulter, Fullerton, Calif.) (41).

### Cell Counts for Peripheral Blood Cell

250 μL peripheral blood samples of each animal were obtained from inferior vena cava in heparinized syringes when they were sacrificed. Cell counts were performed by automatic analyzing system (Celldyne, Abbott Lab, Abbott Park, Ill.), as well as manual counting for WBC differential.

### Renal Function and Structural Analysis

Kidneys were fixed with formaldehyde and stained with hematoxylin-eosin stain. An established tubular injury score was used (41): grade 0 for less than 5% of the region to have necrosis, grade 1 for from 5% to less than 25%, grade 2 for from 25% to less than 50%, grade 3 for from 50% to less than 75% and grade 4 for above 75%. Serum creatinine levels were used as a marker of renal function, and analyzed by the Jaffe method using creatinine reagents (SIGMA, St. Louis, Mo.) and an autoanalyzer (COBAS FARA Roche Diagnostics Corp., Indianapolis, Ind.).

### Statistical Analysis

Means and standard deviations were calculated. One-way ANOVA analysis was used for comparison, and statistical significance was determined as P<0.05.

### Results

### The Effect of Depleting Ab's on Renal IRI.

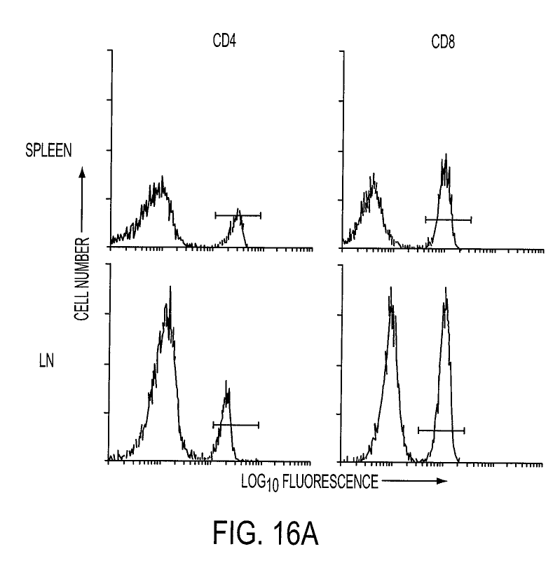

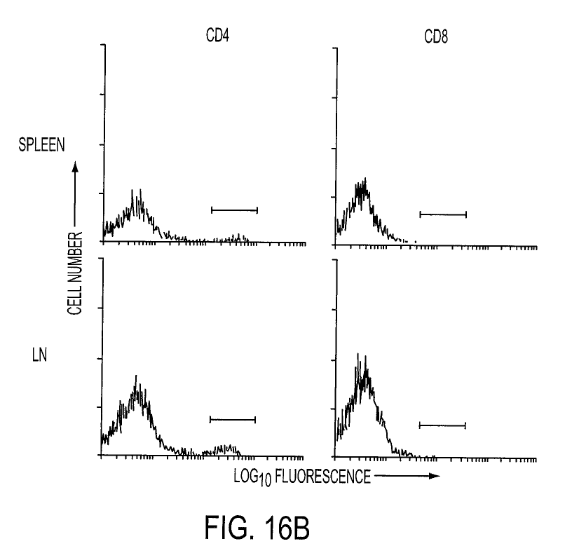

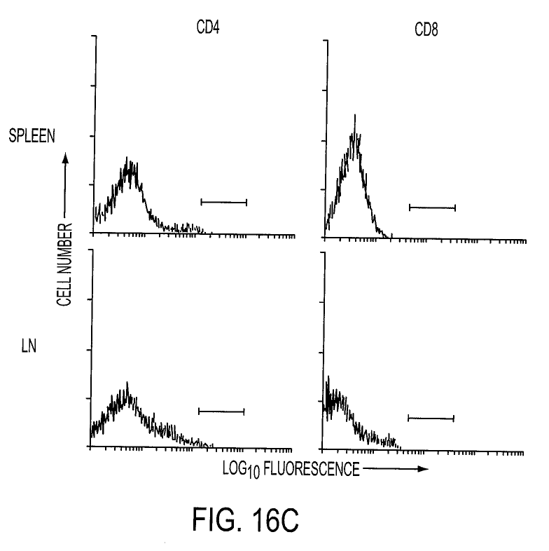

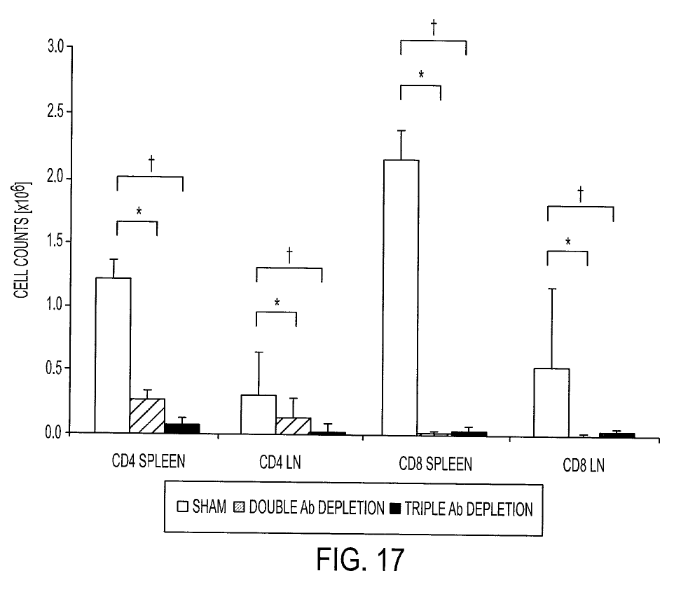

Mice underwent treatment with PBS, double or triple Ab depletion followed by renal IRI. Saturating amounts of antibody was administered based on both previous studies with these antibodies in transplant rejection models as well as extensive dosing and timing data from our own lab. Preliminary studies with CD4 Ab or CD8 Ab alone did not show any tissue protection from renal IRI despite significant depletion of each individual subset (data not shown). When CD4 Ab was combined with CD8 Ab, no significant protection from renal IRI was observed compared with sham thymectomized PBS treated group (FIG. 2). The T cells counts from spleen and lymph nodes showed significant depletion of T cells after GK1.5 and 2.43 compared to PBS treated mice (FIGS. 16 and 17).

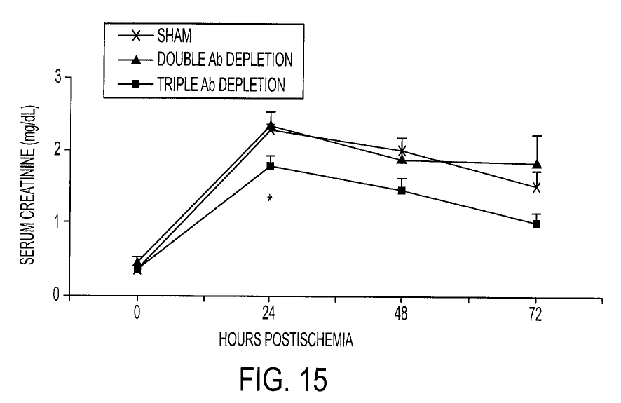

With triple Ab depletion, CD4 and CD8 Ab plus 30.H12, another Ab used to experimentally deplete T cells, a further decrease occurred in total T cells, particularly CD4+ cells. (FIGS. 16c and 17). Renal function after IRI was significantly improved in the triple Ab treated group compared to double Ab and sham treatment (FIG. 15). However, when 30.H12 was administered individually, no renal protection was seen after IRI.

### Ab Depletion Specifically Depleted T Cells

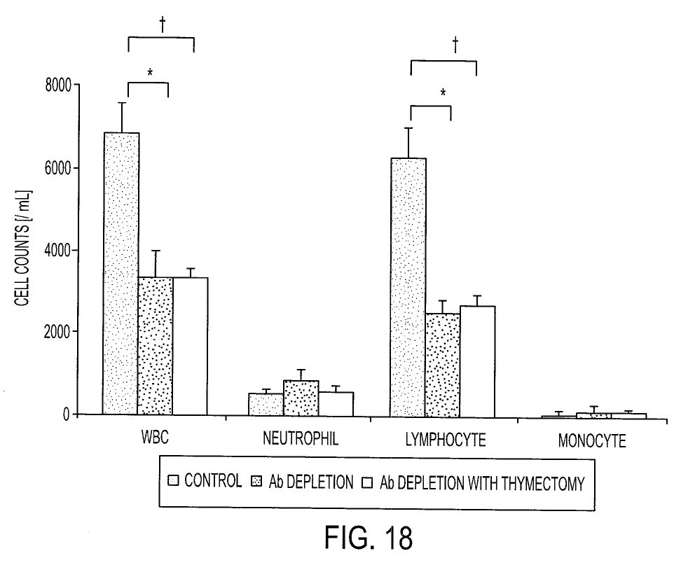

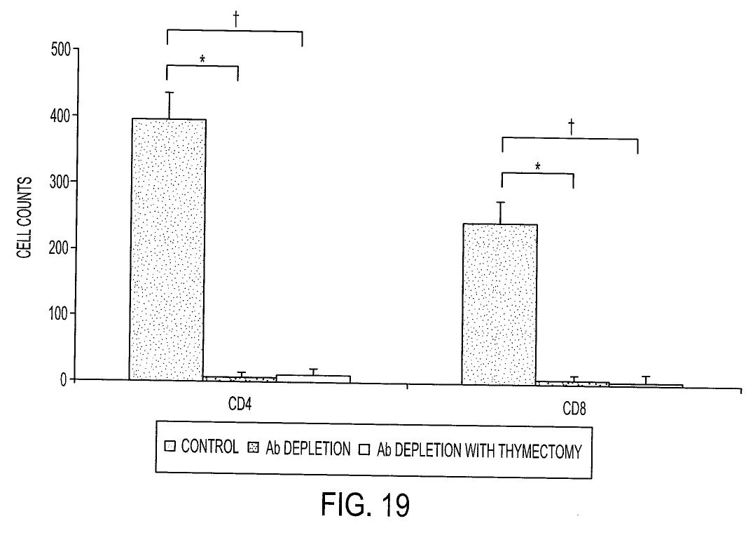

Evaluation of peripheral blood cells showed that numbers of white blood cells, mainly lymphocytes, were significantly reduced after triple Ab depletion, and no effects were seen on the numbers of neutrophils or monocytes (FIG. 18). Flow cytometric analysis of peripheral blood cells demonstrated that triple Ab depletion reduced both CD4 as well as CD8 T cells (FIG. 19). Thymectomy did not play a significant role in influencing T cell numbers with triple Ab depletion (FIGS. 18 and 19).

### Reduction of Tubular Necrosis With Severe T Cell Depletion by Anti-Thy1.2, Anti-CD4 and Anti-CD8 Ab Depletion

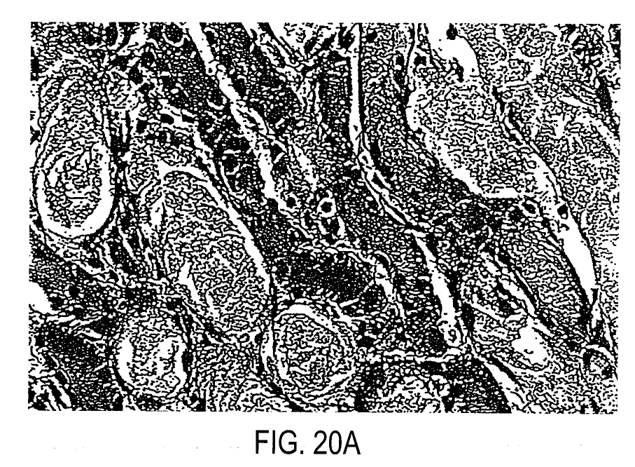

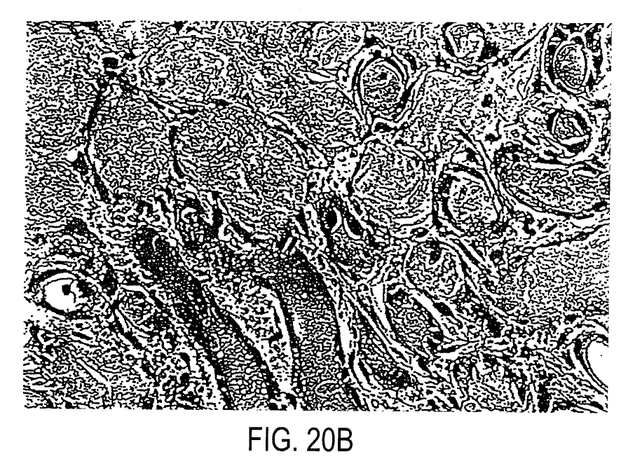

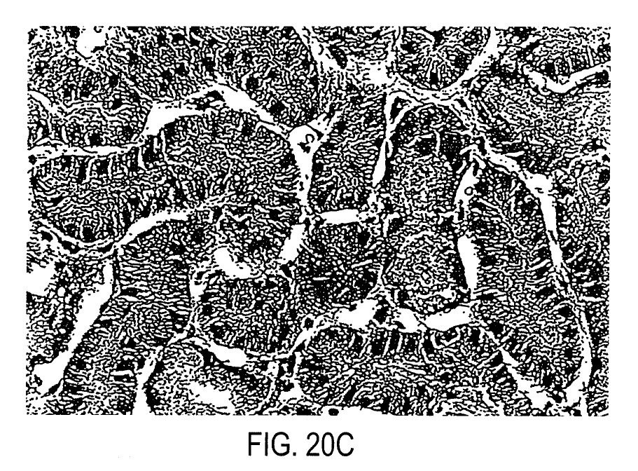

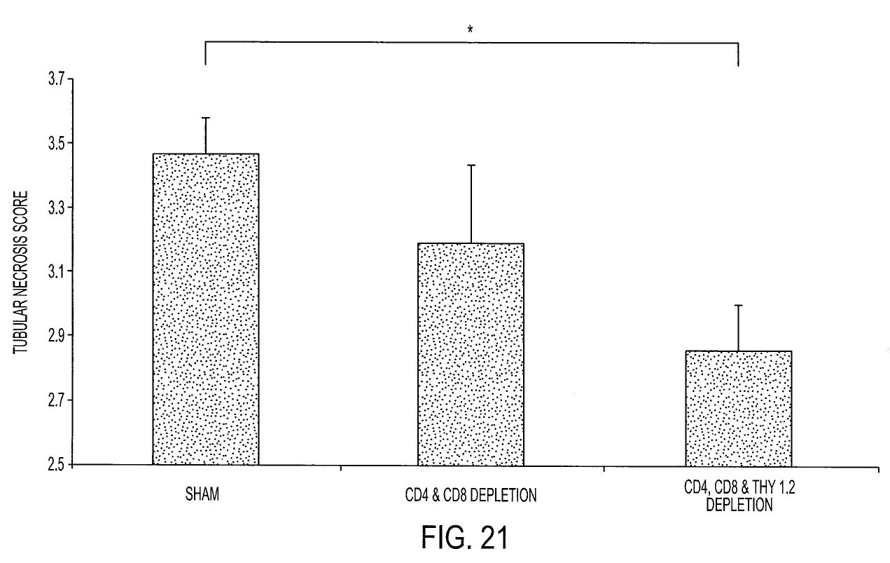

The degree of tubular injury caused by renal IRI in the separate groups was compared in a blinded fashion (FIG. 20). The tubular necrosis score of the triple Ab depletion was significantly reduced compared to double Ab treatment and sham treatment (FIG. 21).

### Discussion

Our results demonstrate that T cell depletion results in protection from renal IRI in wild type mice, consistent with studies demonstrating a key role for T cells in renal IRI using mutant mice which are genetically deficient in T cells. However, we found that very pronounced T cell depletion with combination antibodies was required to be effective.

T cells are a newly recognized mediator of renal IRI, as well as IRI of other organs including liver (42). Studies using T cell deficient mice have demonstrated that mice deficient in both CD4 and CD8 cells, CD4 cells, and athymic mice lacking T cells are all protected from renal IRI (34,35). Furthermore, adoptive transfer of wild type T cells into these mutant mice restores injury phenotype. Despite the pronounced protection observed in mutant mice, these studies are limited by the possibility that abnormalities exist independent of the targeted mutation. Thus, prior to designing a clinical trial, proof of concept in wild type animals is important supportive data. The present observation that Ab administration designed to deplete T cells decreases renal structural and functional derangement after ischemia reperfusion demonstrates that the important role for T cells in renal IRI extends beyond knockouts to wild type animals. The fact that both function and structures were protected, which does not always occur in synchrony, is evidence for a significant role of T cells in this process. However, enthusiasm for this approach is limited by the extensiveness of the T cell depletion, which may be very challenging in humans.

Our preliminary work in this model led to confusion as to the efficacy of T cell depletion in renal IRI, because individual depletions of CD4+ or CD8+ cells by Abs that clearly reduced T cell numbers, and were effective in allograft rejection models, were not protective. A combination strategy was then undertaken. Ab GK1.5 and 2.43 depleted T cells but was insufficient to protect renal structure or function. However, the addition of a third Ab, 30.H12, which is used to deplete T cells targeting the Th1.2 antigen, decreased T cells (particularly CD4+ cells) further and led to renal protection. We entertained the possibility that the 30.H12 Ab was protective in renal IRI on its own, however, administration of this Ab alone did not protect from renal IRI. We cannot exclude the possibility that this Ab had effects independent of purely cellular depletion, such as signaling. A non-specific effect of Ab is unlikely given that various combinations of Abs in high doses had no effect on renal outcome.

We evaluated the possibility that our aggressive T cell depletion reduced other leukocyte populations. However, no significant changes in neutrophils or monocyte counts were seen. In addition, we acknowledge that thymectomy itself is the aggressive procedure and does not reflect a common clinical situation. We performed Ab depletion in the absence and presence of thymectomy, and found that the effects were comparable.

The mechanisms of T cell involvement in renal IRI are not known and were not addressed in this study. However, renal IRI has a complex pathophysiology with involvement of multiple processes including cytokines, neutrophils, complement, growth factors, apoptosis and deranged vascular reactivity (26-28). Since few T cells are seen in renal IRI, but the functional effects are pronounced, it is likely that T cells are working in conjunction with other factors, rather than working alone in mediating tubular toxicity.

Our finding that aggressive T cell depletion can improve the course of renal IRI has implications for future studies. Current clinical depletion strategies, including the use of OKT3, may not be adequate to deplete T cells for clinical benefit in IRI. Small molecules or drugs directed to T cell function may be a more useful strategy, as preliminary evidence suggests with mycophenolic acid's beneficial role in a rat model of renal IRI (43). In addition, if a small population of T cells is adequate to participate in the pathogenesis of renal IRI, it will be important to carefully characterize this population.

### EXAMPLE 3

Whole body ischemia reperfusion with subsequent kidney injury is a common cause of acute renal failure (ARF). However, most experimental work has used an isolated clamp model of renal ischemia reperfusion injury (IRI), which may not reflect whole body IRI events. This may, in part, contribute to discrepancies between results in animal models of ARF and outcomes of clinical trials. We therefore evaluated kidney responses after whole body ischemia reperfusion in mice. Mice underwent 10 min of potassium chloride-induced cardiac arrest followed by cardiopulmonary resuscitation (CPR) or sham arrest (SA). Serum creatinine (SCr) was measured at 24 and 72 h post-arrest. Kidneys were analyzed for tubular injury and myeloperoxidase (MPO) levels. SCr (mg/dL) was significantly increased at 24 and 72 h in mice after CPR (1.52±0.30 and 0.62±0.05 respectively, p<0.05) compared to SA (0.55±0.03 and 0.35±0.05). CPR mice also showed extensive tubular injury compared to SA. Renal MPO levels significantly increased at 24 h in mice following CPR compared to SA. Based on recent data that has implicated T cells in the pathogenesis of isolated organ IRI, we hypothesized that T cells could also mediate whole body IRI. Wild type mice were compared to T cell deficient (nu/nu) mice. T cell deficient mice had significantly reduced SCr at 24 and 48 h postischemia (0.92±0.17 and 1.06±0.32 respectively, p<0.05) compared to wild type animals (1.90±0.21 and 3.00±0.65). T cell deficient mice also had reduced tubular injury and improved survival following CPR compared to wild type animals. These data demonstrate that: 1) whole body ischemia for 10 min followed by resuscitation leads to a similar degree of ischemic ARF to 30 min of isolated renal artery clamping, 2) the kidney response following whole body IRI is characterized by marked inflammation, and 3) T cell deficient mice have improved renal outcomes as well as mortality after whole body IRI. This whole body IRI model may more closely simulate changes in human ARF after hypotension compared to the isolated renal clamp approach.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### REFERENCES

1. Molitoris, B. A. 1998. Ischemic acute renal failure: exciting times at our fingertips. Curr. Opin. Nephrol. Hypertens. 7:405-406.
2. Humes, H. D. 1995. Acute renal failure: prevailing challenges and prospects for the future. Kidney Int. 50:S26-S32.
3. Star, R. A. 1998. Treatment of acute renal failure. Kidney Int. 54:1817-1831.
4. Terasaki, P. I., Cecka, J. M., Gjertson, D. W., and Takemoto, S. 1995. High survival rates of kidney transplants from spousal and living unrelated donors. N. Engl. J. Med. 333:333-336.
5. Rabb, H., O'Meara, Y. M., Madema, P., Coleman, P., and Brady, H. R. 1997. Leukocytes, cell adhesion molecules and ischemic acute renal failure. Kidney Int. 51:1463-1468.
6. Solez, K., Morel-Maroger, L., and Sraer, J. D. 1979. The morphology of “acute tubular necrosis” in man: analysis of 57 renal biopsies and a comparison with the glycerol model. Medicine 58:362-376.
7. Olsen, S. and Solez, K. 1979. Acute tubular necrosis and toxic renal injury. In Renal Pathology. C. C. Tisher and B. M. Brenner, editors. J. B. Lippincott Co., Philadelphia. 769-809.
8. Takada, M., Nadeau, K. C., Shaw, G. D., Marquette, K. A., and Tilney, N. L. 1997. The cytokine-adhesion molecule cascade in ischemia/reperfusion injury of the rat kidney. Inhibition by a soluble P-selectin ligand. J. Clin. Invest. 99:2682-2690.
9. Rabb, H., Daniels, F., O'Donnell, M., Haq, M., Saba, S., Keane, W., and Tang, W. 2000. Pathophysiological role of T lymphocytes in renal ischemia reperfusion injury in mice. Am. J. Physiol. 279:F525-F531.
10. Zwacka, R. M., Zhang, Y., Halldorson, J., Schlossberg, H., Dudus, L., and Engelhardt, J. F. 1997. CD4(+) T-lymphocytes mediate ischemia/reperfusion-induced inflammatory responses in mouse liver. J. Clin. Invest. 100:279-289.
11. Horie, Y. Wolf, R., Chervenak, R. P., Jennings, S. R., and Granger, D. N. 1999. T-lymphocytes contribute to hepatic leukostasis and hypoxic stress induced by gut ischemia-reperfusion. Microcirculation 6:267-280.
12. Le Moine O., Louis, H., Demols, A., Desalle, F., Demoor, F., Quertinmont, E., Goldman, M., and Deviere, J. 2000. Cold liver ischemia-reperfusion injury critically depends on liver T cells and is improved by donor pretreatment with interleukin 10 in mice. Hepatology 31:1266-1274.
13. Coulombe, M., Yang, H., Wolf, L. A., and Gill, R. G. 1999. Tolerance to antigen presenting cell-depleted islet allografts is CD4 T cell dependent. J. Immunol. 162:2503-2510.
14. Desai, N. M., Bassiri, H., Kim, J., Koller, B. H., Smithies, O., Barker, C. F., Naji, A., and Markmann, J. F. 1993. Islet allograft, islet xenograft, and skin allograft survival in CD8+ T lymphocyte-deficient mice. Transplantation 55:718-722.
15. Goes, N., Urmson, J., Ramassar, V., and Halloran, P. F. 1995. Ischemic acute tubular necrosis induces an extensive local cytokine response. Evidence for induction of interferon-gamma, transforming growth factor-beta 1, granulocyte-macrophage colony-stimulating factor, interleukin-2, and interleukin-10. Transplantation. 59:565-572.
16. Kokura S., Wolf, R. E., Yoshikawa, T., Ichikawa, H., Granger, D. N., and Aw, T. Y. 2000. Endothelial cells exposed to anoxia/reoxygenation are hyperadhesive to T-lymphocytes: kinetics and molecular mechanisms. Microcirculation 7:13-23.
17. Matzinger, P. 1998. An innate sense of danger. Semin. Immunol. 10:399-415.
18. Lu, C. Y., Penfield, J. G., Kielar, M. L., Vazquez, M. A., and Jeyarajah, D. R. 1999. Hypothesis: Is renal allograft rejection initiated by the response to injury sustained during the transplant process? Kidney Int. 55:2157-2168.
19. Kelly, K. J., Williams, W. W., Colvin, R. B., Meehan, S. M., Springer, T. A., Gutierrez-Ramos, J. C., and Bonventre, J. V. 1996. Intracellular adhesion molecule-1 deficient mice are protected against ischemic renal injury. J. Clin. Invest. 97:1056-1063.
20. Okusa, M. D., Linden, J., Huang, L., Rieger, J. M., Macdonald, T. L., and Huynh, L. P. 2000. A(2A) adenosine receptor-mediated inhibition of renal injury and neutrophil adhesion. Am. J. Physiol. Renal Physiol. 279:F809-F818.

21. Rabb, H., Mendiola, C. C., Dietz, J., Saba, S. R., Issekutz, T. B., Abanilla, F., Bonventre, J. V., and Ramirez, G. 1994. Role of CD11a and CD11b in ischemic acute renal failure in rats. Am. J. Physiol. 267:F1052-F1058.
22. Rabb, H., Ramirez, G., Saba, S. R., Reynolds, D., Xu, J., Flavell, R., and Antonia, S. 1996. Renal ischemia reperfusion injury in L-selectin mice. Am J. Physiol. 271: F408-F413.
23. Ysebaert, D. K., De Greef, K. E., Vercauteren, S. R., Ghielli, M., Verpooten, G. A., Eyskens, E. J., De Broe, M. E. 2000. Identification and kinetics of leukocytes after severe ischemia/reperfusion renal injury. Nephrol. Dial. Transplant 15:1562-1574.
24. Rabb, H., Oliverstein, R., Issekutz, T. B., Renzi, P. M., and Martin, J. G. 1994. The role of the leukocyte adhesion molecules VLA-4, LFA-1, and Mac-1 in allergic airway responses in the rat. Am. J. Resp. Crit Care Med. 5:1186-1191.
25. Rabb, H., and Martin, J. G. 1997. An emerging paradigm shift on the role of leukocyte adhesion molecules. J. Clin. Invest. 12:2937-2938.
26. Molitoris B A. Ischemic acute renal failure: exciting times at our fingertips. Curr Opin Nephrol Hypertens 1998:7: 405-406.
27. Star R A. Treatment of acute renal failure. Kidney Int 1998:54: 1817-1831.
28. Thadhani R, Pascual M, Bonventre J V. Acute renal failure. N Engl J Med 1996:334: 1448-1460.
29. Tilney N L, Guttmann R D. Effects of initial ischemia/reperfusion injury on the transplanted kidney. Transplantation 1997:64: 945-947.
30. Freedland S J, Shoskes D A. Economic impact of delayed graft function and suboptimal kidneys. Transplantation Reviews 1999:13: 23-30.
31. Rabb H, O'Meara Y M, Maderna P, Coleman P, Brady H R. Leukocytes, cell adhesion molecules and ischemic acute renal failure. Kidney Int 1997:51: 1463-1468.
32. Solez K, Morel-Maroger L, Sraer J D. The morphology of “acute tubular necrosis” in man: analysis of 57 renal biopsies and a comparison with the glycerol model. Medicine (Baltimore ) 1979:58: 362-376.
33. Takada M, Chandraker A, Nadeau K C, Sayegh M H, Tilney N L. The role of the B7 costimulatory pathway in experimental cold ischemia/reperfusion injury. J Clin Invest 1997:100: 1199-1203.
34. Rabb H, Daniels F, O'Donnell M et al. Pathophysiological role of T lymphocytes in renal ischemia reperfusion injury in mice. Am J Physiol Renal Physiol 2000:279: F525-F531.
35. Burne M J, Daniels F, El Ghandour A et al. Identification of the CD4(+) T cell as a major pathogenic factor in ischemic acute renal failure. J Clin Invest 2001:108: 1283-1290.
36. Cosimi A B, Colvin R B, Burton R C et al. Use of monoclonal antibodies to T-cell subsets for immunologic monitoring and treatment in recipients of renal allografts. N Engl J Med 1981:305: 308-314.
37. Zhao Y, Sykes M. Resistance to monoclonal antibody-induced CD8+ T-cell depletion in thymectomized MHC class II-deficient mice. Transplantation 1997:64: 489-494.
38. Coligan J E, Kruisbeek A M, Margulies D H, Shevach E M, Strober W. Current Protocols in Immunology. New York: John Wiley & Sons, 1991.
39. Ledbetter J A, Herzenberg L A. Xenogeneic monoclonal antibodies to mouse lymphoid differentiation antigens. Immunol Rev 1979:47: 63-90.
40. Wilson M E, Sandor M, Blum A M et al. Local suppression of IFN-gamma in hepatic granulomas correlates with tissue-specific replication of Leishmania chagasi. J Immunol 1996:15: 2231-2239.
41. Rabb H, Ramirez G, Saba S R et al. Renal ischemic-reperfusion injury in L-selectin deficient mice. Am J Physiol 1996:271: F408-F413.
42. Zwacka R M, Zhang Y, Halldorson J, Schlossberg H, Dudus L, Engelhardt J F. CD4(+) T-lymphocytes mediate ischemia/reperfusion-induced inflammatory responses in mouse liver. J Clin Invest 1997:100: 279-289.
43. Valentin J F, Bruijn J A, Paul L C. Donor treatment with mycophenolate mofetil: protection against ischemia-reperfusion injury in the rat. Transplantation 2000:69: 344-350.

I claim:

1. A method to treat an isolated kidney for transplantation, comprising:
depleting CD4⁺ T cells in blood perfusing the isolated kidney to reduce the level to less than 5% of the untreated level.

2. A method to treat a kidney donor, comprising:
depleting CD4⁺ T cells in the kidney donor to a level which is less than 5% of the untreated level in the kidney donor's peripheral blood.

3. A method to treat a transplant kidney recipient, comprising:
depleting CD4⁺ T cells in the transplant kidney recipient to a level which is less than 5% of the untreated level in the transplant kidney recipient's peripheral blood.

4. A method to prevent or treat acute renal failure in a patient in a patient in need thereof, comprising:
depleting CD4⁺ T cells in a patient to a level which is less than 5% of the untreated level in the patient's peripheral blood, wherein the patient is at risk of or experiencing acute renal failure.

5. The method of any of claims 1-4 wherein the step of depleting is performed using antibodies.

6. The method of any of claims 1-4 wherein the step of depleting is performed using a cocktail of antibodies raised against different antigens.

7. The method of any of claims 1-4 wherein the step of depleting is performed using antibodies to CD4, CD8, and Thy 1.2 antigens.

8. The method of claim 3 wherein the depleting is performed before the kidney is transplanted into the recipient.

9. The method of claim 3 wherein the depleting is performed after the kidney is transplanted into the recipient.

10. The method of claim 4 wherein the patient has diabetes.

11. The method of claim 4 wherein the patient has hypertension.

12. The method of claim 4 wherein the patient has chronic glomerulonephritis.

13. The method of claim 4 wherein the patient has polycystic kidney disease.

14. A method to prevent or treat acute renal failure in a patient in a patient in need thereof, comprising:
modulating CD4⁺ T cell activity in a patient such that level of IFN-γ in the patient's peripheral blood is less than 35% of untreated level, wherein the patient is at risk of or experiencing acute renal failure.

15. A method to treat an isolated kidney for transplantation, comprising:
modulating CD4⁺ T cell activity in blood perfusing the isolated kidney to reduce level of IFN-γ to less than 35% of untreated level.

16. A method to treat a kidney donor, comprising:
modulating CD4⁺ T cell activity in the kidney donor such that the level of IFN-γ is less than 35% of untreated level in the kidney donor's peripheral blood.

17. A method to treat a transplant kidney recipient, comprising:
modulating CD4⁺ T cell activity in the transplant kidney recipient such that the level of IFN-γ is less than 35% of untreated level in the transplant kidney recipient's peripheral blood.

18. The method of claim 17 wherein the modulating is performed before the kidney is transplanted into the recipient.

19. The method of claim 17 wherein the modulating is performed after the kidney is transplanted into the recipient.

20. The method of claim 14 wherein the patient has diabetes.

21. The method of claim 14 wherein the patient has hypertension.

22. The method of claim 14 wherein the patient has chronic glomerulonephritis.

23. The method of claim 14 wherein the patient has polycystic kidney disease.
